SEQUENCE LISTING (2 sequences)

SEQ ID NO: 1 - DNA, 57 bp, Artificial
  neuropeptide Y analog
gcaagatactatagtgcattaagacattatataaatttaataacaagacaaagaaca

SEQ ID NO: 2 - protein, 19 aa, Artificial
  Synthetic Construct
ARYYSALRHYINLITRQRT

(19) United States
(12) Patent Application Publication
(10) Pub. No.: US 2006/0105942 A1
(43) Pub. Date: May 18, 2006

(54) Inhibition of cell division based use of analogs of neuropeptide y

(76) Inventors: Claire Lewis, SHEFFIELD (GB); Carolyn Staton, Sheffield (GB)

Correspondence Address: KLARQUIST SPARKMAN, LLP, 121 SW SALMON STREET, SUITE 1600, PORTLAND, OR, 97204, US

(21) Appl. No.: 10/521,974

(22) PCT Filed: Jul. 18, 2003
(86) PCT No.: PCT/GB03/03089

§ 371 Date: Aug. 11, 2005

(30) Foreign Application Priority Data
Jul. 23, 2002 (GB) 0217015.7

(51) Int. Cl. A61K 38/12; A61K 48/00; C12Q 1/68; C07K 7/64

(52) U.S. Cl. 514/9; 530/317; 514/44; 435/6; 435/69.1; 435/320.1; 435/325

(57) ABSTRACT

We describe peptides with cell-cycle inhibitory and anti-angiogenic activity and their use in the treatment of diseases which would benefit from the inhibition of cell division and/or angiogenesis, for example cancer.

The invention relates to peptides with cell-cycle inhibitory and anti-angiogenic activity and their use in the treatment of diseases which would benefit from the inhibition of cell division and/or angiogenesis, for example cancer.

The eukaryotic cell cycle is divided into distinct phases, G1 (cell growth and preparation for DNA synthesis), S (DNA synthesis) G2 (preparation for mitosis), M (mitosis, nuclear division). The cell-cycle is highly regulated and many diseases are characterised by abnormal cell-division, for example, cancer or psoriasis.

Angiogenesis, the development of new blood vessels from an existing vascular bed, is a complex multistep process that involves the degradation of components of the extracellular matrix and then the migration, cell-division and differentiation of endothelial cells to form tubules and eventually new vessels. Angiogenesis is involved in pathological conditions such as tumour cell growth; non-cancerous conditions such as neovascular glaucoma; inflammation; diabetic nephropathy; retinopathy, rheumatoid arthritis; inflammatory bowel diseases (eg Crohn's disease, ulcerative colitis); and psoriasis.

The vascular endothelium is normally quiescent. However upon activation endothelial cells proliferate and migrate to form microtubules which will ultimately form a capillary bed to supply blood to developing tissues and, of course, a growing tumour. A number of growth factors have been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF); and platelet derived growth factor (PDGF), Folkman, Nature Medicine, 1: 27-31, 1995; Leek et al J. Leuk. Biol., 56: 423-35, 1994.

A further disease characterised by uncontrolled cell division is psoriasis. Psoriasis is a generic term to cover a range of diseases characterized by abnormal proliferation of skin cells. The disease covers the following list which is not exhaustive but merely illustrative: nail psoriasis; scalp psoriasis; plaque psoriasis; pustular psoriasis; guttate psoriasis; inverse psoriasis; erythrodermic psoriasis; psoriatic arthritis.

Psoriasis is one of the most frequent skin diseases, affecting 1-3% of the Caucasian population world wide. The disease is characterised by alterations in a variety of different cell types. These include epidermal keratinocytes which are characterised by hyperproliferation and an altered differentiation which is indicated by focal parakeratosis and aberrant expression of keratinocyte genes encoding hyperproliferation-associated keratin pair 6/16, involucrin, fillagrin, and integrin adhesion molecules (eg VLA-3, 5, 6).

According to an aspect of the invention there is provided a peptide comprising the amino acid sequence ARYYSALRHYINLITRQRT, or a variant peptide wherein said sequence is modified by addition, deletion of substitution of at least one amino acid residue, for use as a pharmaceutical agent.

In a preferred embodiment of the invention there is provided a pharmaceutical composition comprising a peptide according to the invention.

According to an aspect of the invention there is provided a peptide comprising an amino acid sequence, ARYYSALRHYINLITRQRT, or part thereof, or variant thereof which has been modified by addition, deletion or substitution of at least one amino acid residue, wherein said peptide has cell-cycle inhibitory activity, for the manufacture of a medicament for use in the treatment of diseases or conditions which would benefit from the inhibition of cell division.

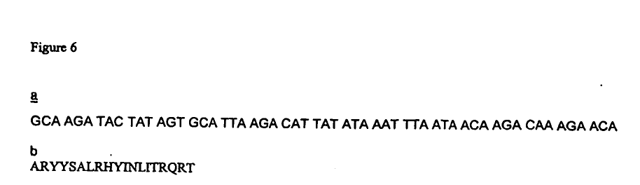

According to a further aspect of the invention there is provided an isolated nucleic acid molecule which encodes a peptide characterised in that the nucleic acid molecule is selected from the following group:
i) a nucleic acid molecule comprising the nucleic acid sequence presented in FIG. 6;
ii) a nucleic acid molecule as represented by the sequence presented in FIG. 6 which has been modified by addition, deletion or substitution of at least one nucleotide base within at least one codon to encode a variant peptide which has cell-cycle inhibitory activity,
iii) a nucleic acid molecule which hybridizes to the sequence in (i) or (ii); and
iv) a nucleic acid molecule comprising a nucleic acid sequence which is degenerate as a result of the genetic code to the sequences identified in (i)-(iii); for the manufacture of a medicament for use in the treatment of diseases or conditions which would benefit from an inhibition of cell-division.

According to a further aspect of the invention there is provided a peptide encoded by the nucleic acid according to the invention for the manufacture of a medicament for use in the treatment of diseases or conditions which would benefit from the inhibition of cell division.

In a further preferred embodiment of the invention said peptide inhibits angiogenesis.

In a preferred embodiment of the invention said disease is selected from the group consisting of: cancer; psoriasis; neovascular glaucoma; rheumatoid arthritis; diabetic retinopathy.

In a further preferred embodiment of the invention said disease in cancer.

In a yet further preferred embodiment of the invention said disease in psoriasis.

Preferably said psoriatic condition is selected from the group consisting of: nail psoriasis; scalp psoriasis; plaque psoriasis; pustular psoriasis; guttate psoriasis; inverse psoriasis; erythrodermic psoriasis; psoriatic arthritis.

Reference to cell-cycle inhibition and anti-angiogenic activity is determined by assays hereindisclosed. For example, the peptides of the invention are tested by in vitro assays which include the inhibition of endothelial cell mediated tubule formation, inhibition of endothelial cell migration, inhibition of VEGF and bFGF induced endothelial cell proliferation and endothelial cell cytotoxicity assays, FACS analysis of cell cultures which have been exposed to peptides of the invention. Peptides can also be tested in vivo using murine tumour models as hereindisclosed.

A variant, (i.e. a fragment peptide and reference peptide) may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Most highly preferred are variants which retain the same biological function and activity as the reference peptide from which it varies.

A peptide according to the invention is a variant wherein one in which one or more amino acid residues are substituted with conserved or non-conserved amino acid residues, or one in which one or more amino acid residues includes a substituent group. Conservative substitutions are the replacements, one for another, among the aliphatic amino acids Ala, Val, Leu and Be; interchange of the hydroxl residues Ser and Thr, exchange of the acidic residues Asp and Glu; substitution between amide residues Asn and Gln; exchange of the basic residues Lys and Arg; and replacements among aromatic residues Phe and Tyr.

In addition, the invention features peptide sequences having at least 75% identity with the peptide sequences as hereindisclosed, or fragments and functionally equivalent peptides thereof. In one embodiment, the peptides have at least 85% identity, more preferably at least 90% identity, even more preferably at least 95% identity, still more preferably at least 97% identity, and most preferably at least 99% identity with the amino acid sequences illustrated herein.

In a preferred embodiment of the invention said peptide comprises an amino acid sequence, or part thereof, consisting of the sequence ARYYSALRHYINLITRQRT. Preferably said peptide is a peptide consisting of the sequence ARYYSALRHYINLITRQRT.

In a further preferred embodiment of the invention said peptide is a fragment ARYYSALRHYINLITRQRT. Preferably said fragment is at least 3 amino acid residues in length, 4 amino acid residues in length, 5 amino acid residues in length or 6 amino acid residues in length, 7 amino acids in length, 8 amino acids in length, 9 amino acids in length, 10, amino acids in length, 11 amino acids in length, 12 amino acids in length, 13 amino acids in length, 14 amino acids in length, 15 amino acids in length, 16 amino acids in length, 17 amino acids in length, or 18 amino acids in length.

In a yet further preferred embodiment of the invention said peptide is at least 20 amino acid residues in length; 21 amino acid residues; 22 amino acid residues; 23 amino acid residues; 24 amino acid residues; 25 amino acid residues; 26 amino acid residues; 27 amino acid residues; 28 amino acid residues; 29 amino acid residues; 30 amino acid residues in length.

In a further preferred embodiment of the invention said peptide is at least 35 amino acid residues in length; at least 40 amino acid residues; at least 45 amino acid residues; at least 50 amino acid residues; at least 60 amino acid residues; at least 70 amino acid residues; at least 80 amino acid residues; at least 90 amino acid residues; at least 100 amino acid residues in length.

It will be apparent to one skilled in the art that modification to the amino acid sequence of peptides according to the invention could enhance the binding and/or stability of the peptide with respect to its target sequence. In addition, modification of the peptide may also increase the in vivo stability of the peptide thereby reducing the effective amount of peptide necessary to inhibit cell-division or angiogenesis or to induce apoptosis. This would advantageously reduce undesirable side effects which may result in vivo. Modifications include, by example and not by way of limitation, acetylation and amidation.

In a preferred embodiment of the invention said peptide sequence is acetylated. Preferably said acetylation is to the amino terminus of said peptide.

In a further preferred embodiment of the invention said peptide sequence is amidated. Preferably said amidation is to the carboxyl-terminus of said peptide.

In a further preferred embodiment of the invention said peptide, or fragment thereof, is modified by both acetylation and amidation.

It will be apparent to one skilled in the art that fragments of a peptide as herein disclosed, are susceptible to modifications such as acetylation and/or amidation.

Alternatively or preferably, said modification includes the use of modified amino acids in the production of recombinant or synthetic forms of peptides according to the invention. It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (eg F, Br, I), hydroxy or C₁-C₄ alkoxy.

Alternatively, fragments could be synthesised de novo and also modified by, for example, cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a preferred embodiment of the invention the peptides according to the invention are modified by cyclisation.

According to a further aspect of the invention there is provided an agent comprising two or more peptides according to the invention wherein said agent has cell-cycle inhibitory activity.

In a preferred embodiment of the invention said two or more peptides are linked by a linker molecule. Preferably said linker molecule is a flexible linker.

In a further preferred embodiment of the invention said agent comprises a plurality of peptides according to the invention. Preferably said agent has 3, 4, 5, 6, 7, 8, 9, or 10 peptides linked together as an oligomeric peptides. Preferably said peptide has greater than 10 peptides according to the invention.

In a further preferred embodiment of the invention said agent is a dimer of two peptides according to the invention.

In a further preferred embodiment of the invention said linker is a peptide linking molecule. In a preferred embodiment of the invention said peptide linking molecule comprises at least one amino acid residue which links at least two peptides according to the invention.

In a further preferred embodiment of the invention said peptide linking molecule comprises at least 2, 3, 4, 5, 6, 7, 8, 9, or 10 amino acid residues. In a further embodiment of the invention said linking molecule comprises more than 10 amino acid residues.

In an alternative embodiment of the invention, the agent is a fusion protein comprising an inframe translational fusion of the peptides according to the invention.

It will be apparent that the invention encompasses the formation oligomeric polypeptides which comprise identical peptides according to the invention, herein referred to as homo-oligomeric agents as well as polypeptides comprising different modified peptides, referred to as hetero-oligomeric agents.

It will be apparent to one skilled in the art that alternative linkers can be used to link peptides, for example the use of chemical protein crosslinkers. For example homo-bifunctional crosslinker such as disuccinimidyl-suberimidate-dihydrochloride; dimethyl-adipimidate-dihydrochloride; 1,5,-2,4 dinitrobenezene or hetero-bifunctional crosslinkers such as N-hydroxysuccinimidyl 2,3-dibromopropionate; 1-ethyl-3-[3-dimethylaminopropyl] carbodiimide hydrochloride; succinimidyl 4-[n-maleimidomethyl]-cyclohexane-1-carboxylate.

According to a further aspect of the invention there is provide the use of an agent according to the invention as a pharmaceutical.

According to a yet further aspect of the invention there is provided a pharmaceutical composition comprising an oligomeric agent according to the invention.

When administered, the pharmaceutical compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The pharmaceutical compositions of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation.

The pharmaceutical compositions used in the foregoing methods of treatment preferably are sterile and contain an effective amount of peptide, oligomeric agent or nucleic acid encoding said peptide or oligomeric agent, for producing the desired response in a unit of weight or volume suitable for administration to a patient.

The doses of peptide, oligomer, or nucleic acid encoding said peptide/oligomer administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

When administered, the therapeutic preparations of the invention are applied in therapeutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention.

Peptide/oligomer polypeptide compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of peptides/oligomer or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables.

Carrier formulation suitable for oral, subcutaneous, intravenous, intramuscular, etc. administrations can be found in Remington's Pharmaceutical Sciences, Mack Publishing Co., Easton, Pa.

In a preferred embodiment of the invention said pharmaceutical compositions modulates angiogenesis. Preferably said modulation is the inhibition of angiogenesis. Preferably said inhibition relates to endothelial cell stimulated angiogenesis.

Alternatively, or preferably, said inhibition is the inhibition of macrophage and/or tumour cell stimulated angiogenesis.

In a further preferred embodiment of the invention said inhibition is mediated by the inhibition of pro-angiogenic factors. Ideally these are either intracellular or cell surface receptors.

Preferably, peptides/oligomers according to the invention can be manufactured by in vitro peptide synthesis using standard peptide synthesis techniques known to those skilled in the art, see for example, Merrifield B, Protein Science (1996), 5: 1947-1951; The chemical synthesis of proteins; Mutter M, Int J Pept Protein Res 1979 March,13(3):274-7 Studies on the coupling rates in liquid-phase peptide synthesis using competition experiments; and Solid Phase Peptide Synthesis in the series Methods in Enzymology (Fields, G. B. (1997) Solid-Phase Peptide Synthesis. Academic Press, San Diego. #9830).

Alternatively, polypeptides comprising the peptides of the invention can be manufactured by recombinant techniques which are well known in the art

According to a further aspect of the invention there is provided a vector, wherein said vector includes a nucleic acid molecule which encodes for peptides and/or oligomers according to the invention.

Alternatively, vector(s) which include nucleic acid encoding said peptides/oligomers can be adapted for recombinant expression.

In a preferred embodiment of the invention said vector is an expression vector adapted for prokaryotic or eukaryotic cell expression. Preferably said eukaryotic vector is adapted for gene therapy.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences and is therefore position independent). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of environmental cues which include, by example and not by way of limitation, intermediary metabolites or environmental effectors.

Promoter elements also include so called TATA box and RNA polymerase initiation selection (RIS) sequences which function to select a site of transcription initiation.

These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors.

Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol m IRL Press, Oxford UK; DNA Cloning: F M Ausubel et al, Current Protocols in Molecular Biology, John Wiley & Sons, Inc.(1994).

In a yet further preferred embodiment of the invention there is provided a gene therapy vector comprising the nucleic acid according to the invention.

It will be apparent to one skilled in the art that the delivery of gene therapy vectors either to endothelial cells or tumour cells target the production of polypeptides according to the invention to the vicinity of the tumour thereby augmenting the anti-angiogenic effect of said polypeptides.

According to a yet further aspect of the invention there is provided a cell transformed/transfected with the nucleic acid according to the invention. Ideally said nucleic acid is the vector according to the invention.

According to yet still a further aspect of the invention there is provided a non-human, transgenic animal characterised in that said animal incorporates a nucleic acid molecule encoding a polypeptide according to the invention into its genome.

It will be apparent to one skilled in the art that the provision of non-human transgenic animals genetically modified by the provision of a transgene(s) encoding polypeptides according to the invention is an alternative source of active polypeptide.

It is well known in the art that transgenic animals can be used to make various therapeutic polypeptides.

According to a further aspect of the invention there is provided a combined preparation comprising a peptide/oligomer according to the invention and at least one cytotoxic agent.

According to a yet further aspect of the invention there is provided a combined preparation comprising a peptide/oligomer according to the invention and at least one further anti-angiogenic agent.

Examples of cytotoxic agents are well known in the art, for example, and not by way of limitation cytotoxins, such as ricin A-chain or diphtheria toxin; antagonists of the key pro-angiogenic factors in tumours (eg VEGF, bFGF, TNF alpha, PDGF) would include neutralising antibodies or receptors for these factors, or tyrosine kinase inhibitors for their receptors (eg. SU5416 for the VEGF receptor, Flk-1/KDR); prodrug activating enzymes such as, human simplex virus-thymidine kinase HSV-TK, which activates the prodrug, ganciclovir when it is then admininistered systemically; chemotherapeutic agents, such as neocarzinostatin; cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; an enediyne such as chalicheamicin or esperamicin; chlorambucil; ARA-C; vindesine; bleomycin; and etoposide.

Examples of anti-angiogenic agents are known in the art and include, by example and not by way of limitation, angiostatin and endostatin. Anti-angiogenic polypeptides and peptides are also disclosed in WO01/88129 and WO02/18440 respectively, which are incorporated by reference. Anti-angiogenic peptides are also disclosed in currently unpublished GB 0203882.6, which is incorporated by reference.

In a further aspect of the invention there is provided a method to treat an animal which would benefit from inhibition of cell-division comprising:
i) administering an effective amount of an agent comprising a peptide/oligomer according to the invention, to an animal to be treated;
ii). monitoring the effects of said agent on the inhibition of cell-division.

In a preferred method of the invention said treatment is the inhibition of tumour development.

It will be apparent that the above combinations of peptides/oligomers and therapeutic agents will also have benefit with respect to the treatment of other conditions/diseases which are dependent on angiogenesis. For example, neovascular glaucoma, rheumatoid arthritis, psoriasis and diabetic retinopathy.

In a yet further alternative method of treatment, said agent is a gene therapy vector according to the invention.

In addition, said nucleic acid encoding a peptide/oligomer according to the invention is provided with, nucleic acid encoding an agent which augments the anti-angiogenic effect of said peptide/oligomer.

According to a yet further aspect of the invention there is provided an imaging agent comprising a peptide/oligomer according to the invention.

It will be apparent to the skilled artisan that peptides according to the invention can be used to target imaging agents to, for example, tumours, to identify developing tumours or to monitor the effects of treatments to inhibit tumour growth. It will also be apparent that the combined therapeutic compositions which comprise both peptides and a further anti-angiogenic agent may be further associated with an imaging agent to monitor the distribution of the combined therapeutic composition and/or to monitor the efficacy of said combined composition.

Methods used to detect imaging agents are well known in the art and include, by example and not by way of limitation, positron emission tomographic detection of F¹⁸ and C¹¹ compounds.

An embodiment of the invention will now be described by example only and with reference to the following figures:

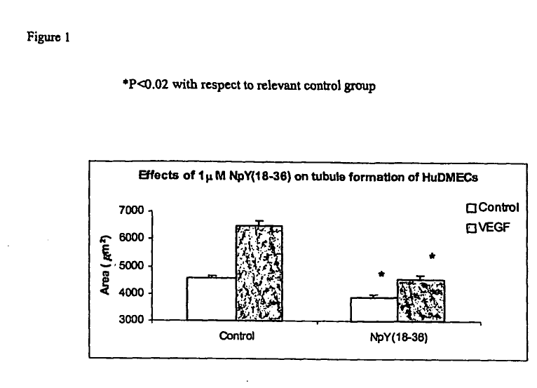

FIG. 1 illustrates the anti-angiogenic effect of peptide Y after 6 hrs incubation;

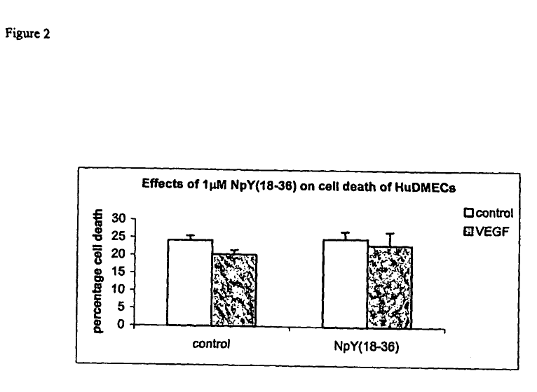

FIG. 2 illustrates the cytoxic effect of peptide Y on in vitro cultures of HuDMEC's;

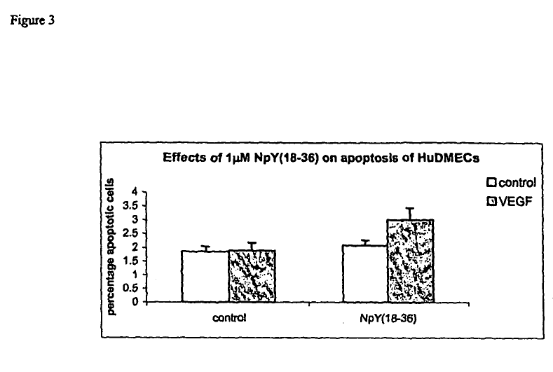

FIG. 3 illustrates the apoptotic effect of peptide Y on in vitro cultures of HuDMEC's;

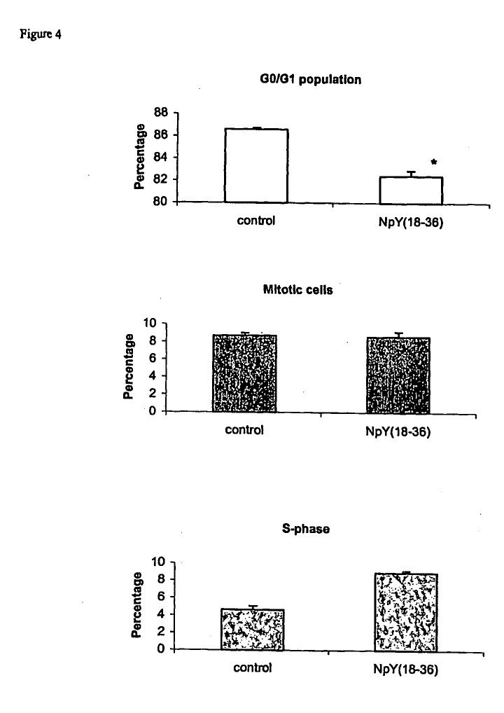

FIG. 4 illustrates the cell-cycle inhibitory effect of peptide Y on in vitro cultures of HuDMEC's;

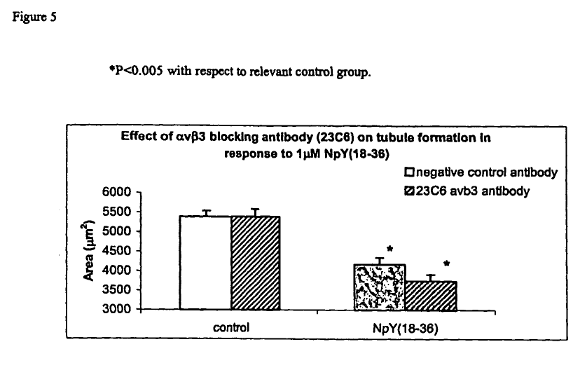

FIG. 5 illustrates the effect of pre-incubation of a αvβ3 antibody on tubule formation of in vitro cultures of HuDMEC's; and

FIG. 6 is the (a) DNA and (b) amino acid sequence of peptide Y.

### EXAMPLE 1

Effects of 6 Hours Exposure to NpY(18-36)

Experiment A: Matrigel

1. Spread 30 μl Matrigel per well and allow to set at room temperature for 15 minutes
2. Lift HuDMECs by trypsinisation, spin down and resuspend in DMEM+1% FCS
3. Seed HuDMECs into Matrigel covered wells (20,000 cell-well) in the presence or absence of VEGF (10 ng/ml) with or without 1 μM NpY(18-36).
4. Allow the plate to stand in the hood without moving for 30 minutes, to enable to cells to attach without ending up round the edges of the wells.
5. Transfer the plate to the incubator (37° C.) for 6 hours
6. Fix the plate with ce-cold 70% ethanol
7. Rinse in water and stain using haematoxylin and eosin, then air dry the plate
8. Analyse two fields of view per well using Scion Image.
   Results are illustrated in FIG. 1.

### EXAMPLE 2

Cytotoxicity

1. Lift cells by trypsinisation and seed into 24 well plates (100,000 cells/well) ip the presence or absence of VEGF and nothing or 1 μM NpY (18-36).
2. Incubate for 6 hours at 37° C.
3. Remove and keep the media in labelled eppendorfs (containing floating, dead cells) and wash the wells with PBS, again adding this to the eppendorfs and centrifuge the eppendorfs for 5 minutes.
4. Lift the remaining cells with trypsin and add to the relevant eppendorfs and spin down.
5. Pour off the supernatant and resuspend the cells in 200 μl and transfer to FACs tubes.
6. Add 25 μl high strength propidium iodide to each tube and read on FACS.
   Results are illustrated in FIG. 2.

### EXAMPLE 3

Annexin V Staining for Apoptosis.

1. HUDMECs were lifted by trypsinisation and seeded at 100,000 cells/well in DMBM+1% FCS over agarose with or without VEGF (10 ng/ml) in the presence or absence of 1 μM NpY(18-36).
2. The cells were incubated for 6 hours at 37° C.
3. After 6 hours the cells were spun down and washed by resuspending in PBS and centrifuing again. This was repeated twice.
4. After the second wash the supernatant was poured off and the cells resuspended in 100 μl Annexin V buffer. 2.5 μl Annexin V solution and 10 μl propidium iodide solution was added per treatment and the cells incubated at room temperature in the dark for 15 minutes,
5. After this time a further 300 μl Annexin V buffer was added to each tube and the cells transferred to FACs tubes for analysis on the FACs within an hour.
   Results are illustrated in FIG. 3.

### EXAMPLE 4

Cell Cycle Analysis
1. Cells were lifted by trypsinisation and plated into 24 well plates at 150,000 cells/well.
2. The cells were treated with nothing (control) or 1 μM NpY(18-36) and incubated for 6 hours.
3. After 6 hours the cells were lifted by trypsinisation (including floating cells) and washed twice in PBS.
4. The cells were fixed in ice-cold 70% ethanol at −20° C. overnight.
5. The cells were centrifuged for 2 minutes, the ethanol supernatant removed and the cells resuspended and washed twice in PBS.
6. The cell pellets were then resuspended in 300 μl PBS containing 1 mg/ml PI and 10 units/ml RNase at room temperature in the dark for a minimum of 2 hours.
7. The samples were then run on the FACs.
   Results are Illustrated in FIG. 4.

### EXAMPLE 5

Matrigel Using αᵥβ₃ Blocking Antibody.

1. Spread 30 μl Matrigel per well and allow to set at room temperature for 15 minutes
2. Lift HuDMECs by trypsinisation, spin down and resuspend in DMEM+1% FCS
3. Seed HuDMECs into Matrigel covered wells (20,000 cells/well) and allow the cells to attach without moving for 30 minutes.
4. After this time either an antibody to αᵥβ₃, 23C6 (10 μg/ml), or a negative control antibody (10 μg/ml) is added to the medium for a further 30 minutes.
5. At this stage 1 μM NpY(18-36) was added to the wells in the presence of 10 ng/ml VEGF and the plate is transfered to the incubator (37° C.) overnight.
6. Fix the plate with ce-cold 70% ethanol
7. Rinse in water and stain using haematoxylin and eosin, then air dry the plate
8. Analyse three fields of view per well using Scion Image.
   Results are illustrated in FIG. 5
   In Vivo Animal Model Studies

Experiments were performed on six-week-old Balb/C mice weighing 15 g, obtained from Sheffield Field Laboratories. All experiments were approved by the Home Office Project Licence Number PPL50/1414.

Tumour Cell Culture

The CT26 cell line was maintained by in vitro passage in Dulbecco's Minimal Eagles Medium containing 10% foetal calf serum, and 1% penicillin and streptomycin and maintained at 37° C. in humidified atmosphere of 5% CO₂ in air. The cell line was routinely checked to ensure freedom from mycoplasma (Mycoplasma rapid detection system, Gena-Probe Incorporated, U.S.A).

Subcutaneous Tumour Implantation

Animals were anaesthetised with an intraperitoneal injection of diazepam (0.5 mg/ml, Dumex Ltd.) and hypnorm (fentanyl citrate 0.0315 mg/ml and fluanisone 1 mg/ml, Janssen Pharmaceutical Ltd.) in the ratio of 1:1 at a volume of 0.1 ml/200 g body weight, with supplementation as required to maintain adequate anaesthesia Naïve Balb/c mice were immunised s.c into the right flank, following removal of the fur. Tumour cells were injected at a concentration of 3×10⁵ viable CT26 cells per animal suspended in 100 ul serum free medium. Animals were then allowed to recover. Tumour growth and animal weights were monitored daily.

Administration of Peptides

Tumour growth was measured daily and when the majority of animals in the cohort had tumour volumes of >100 mm³ but <350 mm³ animals were divided into experimental and control groups. This occurred between 14 and 18 days following implantation of the tumour cell suspension. Animals then received an intraperitoneal (ip) injection of either active drug (peptide at 100 mM; 100 μl) or vehicle (phophate buffered saline, 100 μl). Daily injections continued until the tumour growth in the control animals reached the maximum burden allowed by Home Office legislation.

Assessment of Tumour Growth

Tumour volumes were assessed by calliper measurements of the perpendicular diameters and volumes estimated using the equation:—
Volume=(a²×b)/2
where a is the smaller and b the larger diameter.

Animals were weighed on a daily basis and the general well being monitored.

Statistical Analysis.

All experiments were performed at least three times and data analysed using the Mann-Whitney U test, a non-parametric test that does not assume a Gaussian distribution in the data being analysed. P<0.05 was taken as significant.

1. An isolated nucleic acid molecule which encodes a peptide that can inhibit cell division, wherein the nucleic acid molecule is:
i) a nucleic acid molecule comprising the nucleic acid sequence presented in SEQ ID NO: 1;
ii) a nucleic acid molecule as represented by the sequence presented in SEQ ID NO: 1 which has been modified by addition, deletion or substitution of at least one nucleotide base within at least one codon to encode a variant peptide which has cell-cycle inhibitory activity;
iii) a nucleic acid molecule which hybridizes to the sequence in (i) or (ii); or
iv) a nucleic acid molecule comprising a nucleic acid sequence which is degenerate as a result of the genetic code to the sequences identified in (i)-(iii).

2. A peptide encoded by the nucleic acid according to claim 1, wherein the peptide can inhibit cell division or angiogenesis.

3. (canceled)

4. The peptide according to claim 2, wherein the peptide can treat cancer; psoriasis; neovascular glaucoma; rheumatoid arthritis; or diabetic retinopathy.

5.-6. (canceled)

7. The peptide according to claim 4, wherein said psoriatic condition is nail psoriasis; scalp psoriasis; plaque psoriasis; pustular psoriasis; guttate psoriasis; inverse psoriasis; erythrodermic psoriasis; or psoriatic arthritis.

8. The peptide of claim 2, wherein said peptide comprises the amino acid sequence ARYYSALRHYINLITRQRT (SEQ ID NO: 2).

9. The peptide according to claim 8, wherein said peptide is a peptide consisting of the amino acid sequence ARYYSALRHYINLITRQRT (SEQ ID NO: 2).

10. The peptide of claim 2, wherein said peptide is a fragment of ARYYSALRHYINLITRQRT (SEQ ID NO: 2).

11. The peptide of claim 2, wherein said peptide is acetylated.

12. The peptide according to claim 11, wherein said acetylation is to an amino terminus of said peptide.

13. The peptide of claim 2, wherein said peptide is amidated.

14. The peptide according to claim 13, wherein said amidation is to a carboxyl-terminus of said peptide.

15. The peptide of claim 2, wherein said peptide is modified by both acetylation and amidation.

16. The peptide of claim 2, wherein said peptide is modified by cyclisation.

17. An agent comprising two or more peptides of claim 2, wherein said agent has cell-cycle inhibitory activity.

18. The agent of claim 17, wherein said two or more peptides are linked by a linker molecule.

19. The agent of claim 17, wherein said agent comprises a plurality of peptides.

20. The agent of claim 19, wherein said agent comprises 3, 4, 5, 6, 7, 8, 9, or 10 peptides linked together as an oligomeric peptide.

21. The agent of claim 17, wherein said peptide has greater than 10 peptides.

22. The agent of claim 17, wherein said agent is a dimer of two peptides.

23. The agent of claim 18, wherein said linker is a peptide linking molecule.

24. The agent of claim 23, wherein said peptide linking molecule comprises at least one amino acid residue which links at least two peptides.

25. The agent of claim 24, wherein said peptide linking molecule comprises at least 2, 3, 4, 5, 6, 7, 8, 9, or 10 amino acid residues.

26. The agent of claim 23, wherein said linking molecule comprises more than 10 amino acid residues.

27. The agent of claim 17, wherein said agent is a fusion protein comprising an inframe translational fusion.

28. (canceled)

29. A pharmaceutical composition comprising the agent of claim 17.

30. A vector comprising the nucleic acid molecule of claim 1.

31. A cell transformed/transfected with the nucleic acid molecule of claim 1.

32. A non-human, transgenic animal comprising the nucleic acid molecule of claim 1.

33. A combined preparation comprising the peptide of claim 2 and at least one cytotoxic agent.

34. A combined preparation comprising the peptide of claim 2 and at least one anti-angiogenic agent.

35. A method to treat an animal which would benefit from inhibition of cell-division comprising:
administering an effective amount of the peptide of claim 2 to the animal;
monitoring effects of said peptide on the inhibition of cell-division.

36. The method of claim 35 wherein said treatment is inhibition of tumour development.

37. A method of treating an animal which would benefit from inhibition of cell-division, comprising administering an effective amount of the of claim 35, nucleic acid molecule of claim 1 to the animal.

38. An imaging agent comprising the peptide of claim 2.

39. A peptide comprising the amino acid sequence ARYYSALRHYINLITRQRT (SEQ ID NO: 2), or a variant peptide wherein said sequence is modified by addition, deletion of substitution of at least one amino acid residue.

40. A pharmaceutical composition comprising the peptide of claim 39.
